(19) United States
(12) Patent Application Publication
(10) Pub. No.: US 2024/0366226 A1
(43) Pub. Date: Nov. 7, 2024

(54) BRAIDED ANEURYSM TREATMENT DEVICE WITH FLEXIBLE INVERSION REGION

(71) Applicant: DEPUY SYNTHES PRODUCTS, INC., RAYNHAM, MA (US)

(72) Inventors: LACEY GOROCHOW, MIAMI, FL (US); RUIJIAO XU, MIAMI LAKES, FL (US)

(21) Appl. No.: 18/639,064

(22) Filed: Apr. 18, 2024

Related U.S. Application Data
(60) Provisional application No. 63/464,237, filed on May 5, 2023.

(51) Int. Cl. A61B 17/12; A61B 17/00

(52) U.S. Cl. CPC A61B 17/12113; A61B 17/12145; A61B 2017/00526; A61B 2017/00778

(57) ABSTRACT

A braided aneurysm treatment device that includes a tubular braid. The tubular braid includes an open end, a pinched end, and a predetermined shape. In the predetermined shape the tubular braid includes a first segment extending from the open end to a first inversion, a second segment extending from the first inversion to a second inversion, and a third segment surrounded by the second segment and extending from the second inversion to the pinched end. The strands of the tubular braid include a smaller diameter at the first inversion compared to a strand diameter of the first segment and a strand diameter of the second segment to facilitate folding of the tubular braid at the first inversion when the braid is implanted.

### CROSS-REFERENCE TO RELATED APPLICATIONS

The present application claims the benefit of U.S. Provisional Application No. 63/464,237, filed May 5, 2023, the disclosure of which is hereby incorporated by reference herein.

### FIELD

The present invention generally relates to medical instruments, and more particularly, to embolic implants for aneurysm therapy.

### BACKGROUND

Cranial aneurysms can be complicated and difficult to treat due to their proximity to critical brain tissues. Prior solutions have included endovascular treatment whereby an internal volume of the aneurysm sac is removed or excluded from arterial blood pressure and flow. Current alternatives to endovascular or other surgical approaches can include intravascularly delivered treatment devices that fill the sac of the aneurysm with embolic material or block the entrance or neck of the aneurysm. Both approaches attempt to prevent blood flow into the aneurysm. When filling an aneurysm sac, the embolic material clots the blood, creating a thrombotic mass within the aneurysm. When treating the aneurysm neck, blood flow into the entrance of the aneurysm is inhibited, inducing venous stasis in the aneurysm and facilitating a natural formation of a thrombotic mass within the aneurysm.

Current intravascularly delivered devices typically utilize multiple embolic coils to either fill the sac or treat the entrance of the aneurysm. Naturally formed thrombotic masses formed by treating the entrance with embolic coils can result in improved healing compared to aneurysm masses packed with embolic coils because naturally formed thrombotic masses can reduce the likelihood of distention from arterial walls and facilitate reintegration into the original parent vessel shape along the neck plane. However, embolic coils delivered to the neck of the aneurysm can potentially have the adverse effect of impeding the flow of blood in the adjoining blood vessel, particularly if the entrance is overpacked. Conversely, if the entrance is insufficiently packed, blood flow can persist into the aneurysm. Treating certain aneurysm morphology (e.g. wide neck, bifurcation, etc.) can require ancillary devices such a stents or balloons to support the coil mass and obtain the desired packing density. Once implanted, the coils cannot easily be retracted or repositioned. Furthermore, embolic coils do not always effectively treat aneurysms as aneurysms treated with multiple coils often recanalize or compact because of poor coiling, lack of coverage across the aneurysm neck, blood flow, or large aneurysm size.

Alternatives to embolic coils are being explored, for example a tubular braided implant is disclosed in U.S. Pat. No. 10,751,066 and 10,653,435, incorporated by reference as if set forth in their entireties herein. Tubular braided implants have the potential to easily, accurately, and safely treat an aneurysm or other arterio-venous malformation in a parent vessel without blocking flow into perforator vessels communicating with the parent vessel. Compared to embolic coils, however, tubular braided implants are a newer technology, and there is therefore capacity for improved geometries, configurations, delivery systems, etc. for the tubular braided implants. For instance, the implanted shape and final position of the braid is controlled via manipulation of the proximal ends of a delivery system and a microcatheter, and it can be difficult to arrive at a desired implanted shape and position.

There is therefore a need for improved methods, devices, and systems for implants for aneurysm treatment.

### SUMMARY

In some examples, a tubular braid is disclosed for treating aneurysms. The tubular braid can include an open end, a pinched end, and a predetermined shape. In the predetermined shape the tubular braid can include a first segment extending from the open end to a first inversion, a second segment extending from the first inversion to a second inversion, and a third segment surrounded by the second segment and extending from the second inversion to the pinched end. The strands of the tubular braid can include a smaller diameter at the first inversion compared to a strand diameter of the first segment and a strand diameter of the second segment.

In some examples, the material of the tubular braid at the first inversion can be weakened by oxide removal.

In some examples, the strands of the tubular braid can include a layer of oxide wherein the layer of oxide can be thinner at the first inversion compared to an oxide layer of the first segment and an oxide layer of the second segment.

In some examples, the smaller diameter of the strands at the first inversion can be configured to induce folding of the tubular braid at the first inversion during implantation of the implant.

In some examples, the tubular braid can be configured to expand from a predetermined shape into an implanted shape wherein the thinner oxide layer at the first inversion is configured to induce folding of the tubular braid, thereby allowing this expansion.

In some examples, a method of manufacturing a tubular braid is disclosed. The method can include: shaping a tubular braid to a predetermined shape; heat setting the tubular braid in the predetermined shape in an inert environment; and reducing the braid thickness at the first inversion while retaining braid thickness in portions of the first segment and the second segment. Shaping the tubular braid to the predetermined shape can include: inverting the tubular braid to form a second inversion, shaping an third segment of the tubular braid extending from the second inversion to a pinched end of the tubular braid, inverting the tubular braid to form a first inversion by moving an open end of the tubular braid over at least a portion of the braid, shaping a second segment of the tubular braid extending between the first inversion and the second inversion, positioning the second segment to surround the third segment, and shaping an first segment of the tubular braid extending between the open end and the first inversion.

In some examples, reducing braid thickness at the first inversion while retaining thickness in portions of the first segment and the second segment can include masking at least a portion of the first segment and at least a portion of the second segment while exposing the first inversion.

In some examples, reducing braid thickness at the first inversion while retaining thickness in portions of the first segment and the second segment can include chemically etching the tubular braid.

In some examples, reducing braid thickness at the first inversion while retaining thickness in portions of the first segment and the second segment can include electropolishing the tubular braid.

In some examples, reducing braid thickness at the first inversion while retaining thickness in portions of the first segment and the second segment can result in material of the tubular braid being less rigid at the first inversion compared to the first segment and the second segment in order to induce folding of the tubular braid at the first inversion during implantation of the implant.

In some examples, a method of manufacturing a tubular braid is disclosed. The method can include: shaping a tubular braid of thicker wire to a predetermined shape; and reducing the braid thickness at the first inversion while retaining braid thickness in portions of the first segment and the second segment. Shaping the tubular braid of thicker wire to the predetermined shape can include: inverting the tubular braid to form a second inversion, shaping an third segment of the tubular braid extending from the second inversion to a pinched end of the tubular braid, inverting the tubular braid to form a first inversion by moving an open end of the tubular braid over at least a portion of the braid, shaping a second segment of the tubular braid extending between the first inversion and the second inversion, positioning the second segment to surround the third segment, and shaping an first segment of the tubular braid extending between the open end and the first inversion.

In some examples, reducing braid thickness at the first inversion while retaining thickness in portions of the first segment and the second segment can include chemically etching the tubular braid.

In some examples, reducing braid thickness at the first inversion while retaining thickness in portions of the first segment and the second segment can include masking at least a portion of the first segment and at least a portion of the second segment while exposing the first inversion.

In some examples, reducing braid thickness at the first inversion while retaining thickness in portions of the first segment and the second segment can include chemically etching the tubular braid again after at least a portion of the first segment and at least a portion of the second segment have been masked.

In some examples, reducing braid thickness at the first inversion while retaining thickness in portions of the first segment and the second segment can result in material of the tubular braid being less rigid at the first inversion compared to the first segment and the second segment in order to induce folding of the tubular braid at the first inversion during implantation of the implant.

In some examples, a method of manufacturing a tubular braid is disclosed. The method can include: shaping a tubular braid of electropolished wire to a predetermined shape; heat setting the tubular braid in the predetermined shape in an inert environment; and reducing the braid thickness at the first inversion while retaining braid thickness in portions of the first segment and the second segment. Shaping the tubular braid of electropolished wire to the predetermined shape can include: inverting the tubular braid to form a second inversion, shaping an third segment of the tubular braid extending from the second inversion to a pinched end of the tubular braid, inverting the tubular braid to form a first inversion by moving an open end of the tubular braid over at least a portion of the braid, shaping a second segment of the tubular braid extending between the first inversion and the second inversion, positioning the second segment to surround the third segment, and shaping an first segment of the tubular braid extending between the open end and the first inversion.

In some examples, reducing braid thickness at the first inversion while retaining thickness in portions of the first segment and the second segment can include masking at least a portion of the first segment and at least a portion of the second segment while leaving the first inversion exposed.

In some examples, reducing braid thickness at the first inversion while retaining thickness in portions of the first segment and the second segment can include chemically etching the tubular braid.

In some examples, reducing braid thickness at the first inversion while retaining thickness in portions of the first segment and the second segment can include electropolishing the tubular braid.

In some examples, reducing braid thickness at the first inversion while retaining thickness in portions of the first segment and the second segment can include can result in the material of the tubular braid being less rigid at the first inversion compared to the first segment and the second segment in order to induce folding of the tubular braid at the first inversion during implantation of the implant.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is a side view of the tubular braid in a predetermined shape according to the aspects of the present invention.

FIG. 1B is a cross-sectional view of the tubular braid in an implanted shape according to the aspects of the present invention.

FIG. 2A through 2H are side views illustrating an implant having a tubular braid that expands to a predetermined shape similar to as illustrated in FIG. 1A according to the aspects of the present invention.

FIG. 3A through 3F are views illustrating an implant having a tubular braid expanding to an implanted shape within an aneurysm according to the aspects of the present invention.

FIG. 4A is a perspective view of the tubular braid illustrating the region of reduced wire thickness according to the aspects of the present invention.

FIG. 4B is a cross-sectional view illustrating the difference in strand diameter at the inversions of the tubular braid.

FIG. 5 is a flow diagram illustrating a method of manufacture for a tubular braid according to the aspects of the present invention.

FIG. 6 is a flow diagram illustrating a method of manufacture for a tubular braid of thicker wire according to the aspects of the present invention.

FIG. 7 is a flow diagram illustrating a method of manufacture for a tubular braid of electropolished wire according to the aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

As used herein, the terms “thickness” and “diameter” are to be used interchangeably. They are used in reference to the dimensions of the individual strands of the tubular braid. For example, an embodiment of the present invention may include strands of the tubular braid that can be composed of cylindrical wires, flat wires, rectangular wires, etc. However, when the term “radius” is used, this is solely in reference to the overall dimensions of the tubular braid itself and not the individual strands of the tubular braid.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

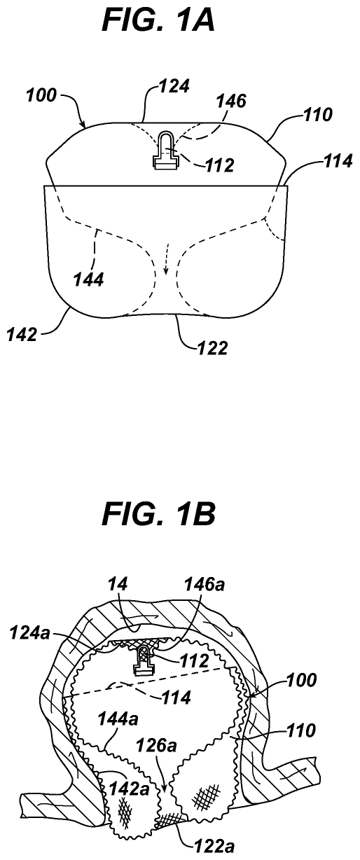

FIG. 1A is an illustration of an implant 100 having a tubular braid 110 in a predetermined shape. The tubular braid can include an open end 114 and a pinched end 112. In the predetermined shape, the tubular braid can include a first segment 142 extending from the open end 114 to a first inversion 122, a second segment 144 extending from the first inversion 122 to a second inversion 124, and a third segment 146 surrounded by the second segment 144 and extending from the second inversion 124 to the pinched end 112. In some examples, the material of the tubular braid 110 can be weakened by oxide removal.

In some examples, the strands 111 of the tubular braid 110 can be configured to induce folding of the tubular braid 110 at the first inversion 122 during implantation of the implant 100. Due to reduced braid 110 thickness and/or oxide removal at the first inversion 122, a physician is able to easily fold the tubular braid into the predetermined shape without damaging the device or the vessel wall. This also allows the device to easily expand from the predetermined shape into the implanted shape. Areas of the braid 110 with reduced thickness and/or oxide removal can have a greater flexibility than areas of the braid 110 having a greater thickness and/or thicker oxide. The difference in flexibility can result in the areas with reduced thickness and/or oxide removal having a greater propensity to form a fold. In the illustrated example, reduced thickness and/or oxide removal at the first inversion 122 results in the braid 110 having a greater propensity to fold at the first inversion 122a compared to a braid 110 having a uniform strand thickness and/or uniform oxide coverage. The resulting fold at the first inversion 122 allows for inversion of the second segment 144 and layering of the second segment 144 against the first segment 142.

In some examples, the tubular braid 110 can be expanded from the predetermined shape (FIG. 1A) into an implanted shape (FIG. 1B) to treat an aneurysm.

FIG. 1B is an illustration of an implant 100 having a tubular braid 110 in an implanted shape. The tubular braid can include an open end 114 and a pinched end 112. In the implanted shape, the tubular braid 110 can include an outer segment 142a contacting the aneurysm's wall, a middle segment 144a nested within the outer segment 142a, an inner segment 146a positioned near the distal portion of the aneurysm wall 14, a proximal inversion 122a positioned at the neck of the aneurysm, and a distal inversion 124a positioned near a distal portion of the aneurysm wall 14. The middle segment 144a also forms a neck opening 126a that is preferably configured with a sufficiently small radius to prohibit blood flow into the aneurysm sac and allow for venous statis.

The implanted shape is based in part on the predetermined shape (FIG. 1A) and the shape of the sac of the aneurysm (or other approximately spherical cavity into which the implant 100 is implanted). The outer segment 142a of the implanted shape corresponds to the first segment 142 of the predetermined shape. The middle segment 144a of the implanted shape corresponds to the second segment 144 of the predetermined shape. The inner segment 146a of the implanted shape corresponds to the third segment 146 of the predetermined shape. The proximal inversion 122a of the implanted shape corresponds to the first inversion 122 of the predetermined shape, and the distal inversion 124a of the implanted shape corresponds to the second inversion 124 of the predetermined shape. Reduced thickness and/or oxide removal at the first inversion 122 results in the braid 110 having a greater propensity to fold at the proximal inversion 122a compared to a braid 110 having a uniform strand thickness and/or uniform oxide coverage. In some examples, the length of the strand thickness of braid 110 at the proximal inversion 122a of the implanted shape will correspond to the path length of the radius of the first inversion zone 122 of the predetermined shape. Further, for the sake of manufacturing, reducing wire thickness at the inversion zone as well as the proximal inversion zone will be sufficient in order to achieve the desired effect of braid 110 having a greater propensity to form a fold in the inversion zones resulting in the device being able to expand easily.

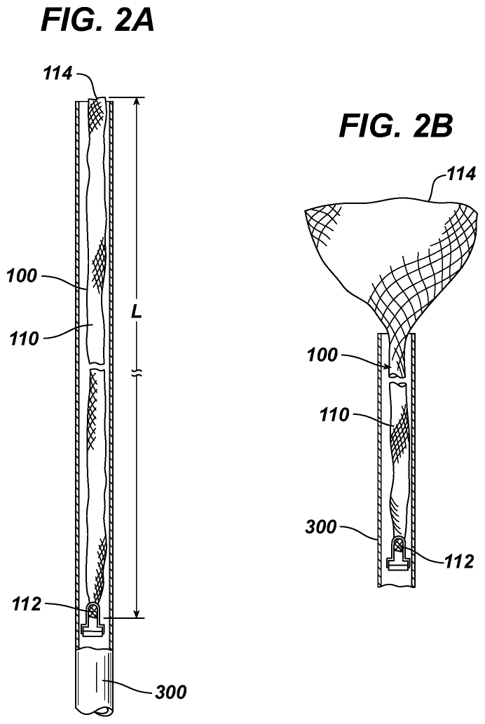

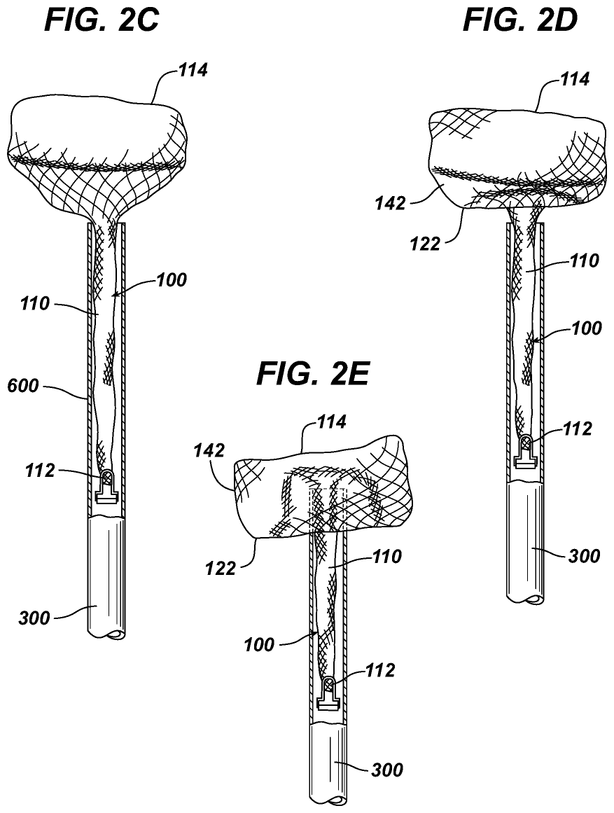

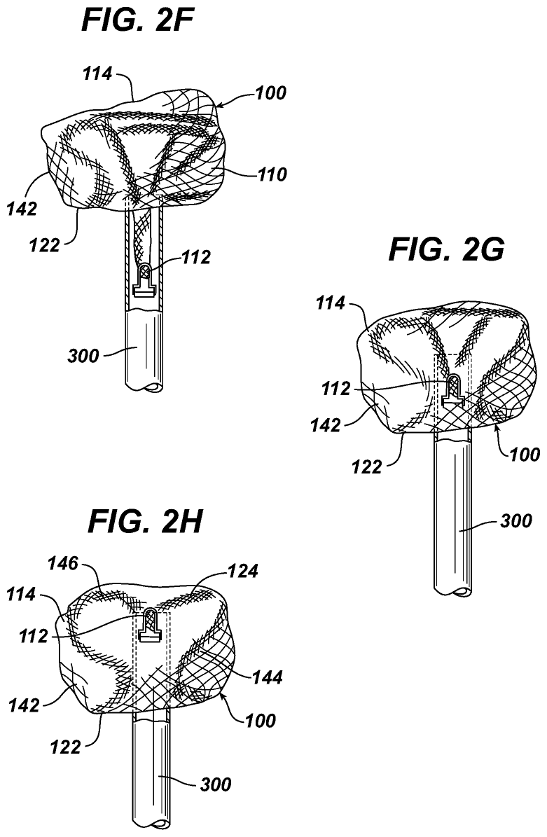

FIG. 2A through FIG. 2H are illustrations of an implant 100 having a tubular braid 110 expanding into a predetermined shape similar to FIG. 1A.

FIG. 2A is an illustration of the implant 100 having the tubular braid 110 configured to a delivery shape within a microcatheter 300 of length L. The tubular braid can include a pinched end 112 positioned near the proximal end of an implant 100 and an open end 114 positioned near a distal end of an implant 100. As illustrated, the braid 110 can be delivered as a single layer such that the delivery shape is an elongated, tubular single layer.

FIG. 2B and FIG. 2C are illustrations of the implant 100 with the open end 114 of the tubular braid 110 positioned to exit the microcatheter 300. The open end 114 expands as the braid 110 is pushed distally out of a distal end of the microcatheter 300.

FIG. 2D is an illustration of the implant 100 in which the tubular braid 110 is expanded after being pushed further distally from the distal end of the microcatheter 300 to form a first inversion 122, defining the first segment 142. In some examples, the tubular braid 110 can be configured to induce folding of the tubular braid 110 at the first inversion 122 during implantation of the implant 100. Due to reduced braid 110 thickness and/or oxide removal at the first inversion 122, a physician is able to easily fold the tubular braid into an implanted shape which resembles the predetermined shape without damaging the device or the vessel wall. This also allows the device to easily expand from the delivery shape into the predetermined shape and provides the physician with freedom to switch between the two if necessary.

FIG. 2E through FIG. 2G illustrate further expansion of the tubular braid 110 as it exits the microcatheter 300. Because the braid 110 has a tendency to fold at the first inversion 122, the braid 110 naturally forms the first inversion 122, which causes the braid to invert and form the second segment 144. The first inversion 122 has a stable position along the length of the braid 110 as the second segment 144 is being pushed out of the distal end of the microcatheter 300. The stable position of the first inversion 122 allows for more precise control of the proximal inversion when the implant 100 is implanted in a spherical cavity such as an aneurysm.

FIG. 2H is an illustration of when all, or nearly all, of the tubular braid 110 exits the microcatheter 300, allowing the second inversion 124, second segment 144, and third segment 146 to form to the predetermined shape. In the illustrated predetermined shape, the first segment 142 extends from the open end 114 to the first inversion 122; the second segment 144 extends from the first inversion 122 to the second inversion 124; and the third segment 146 is surrounded by the second segment 144 and extends from the second inversion 124 to the pinched end 112.

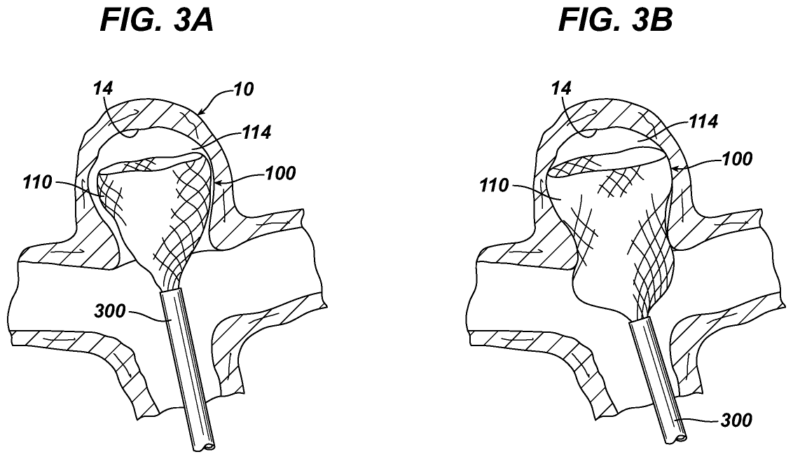

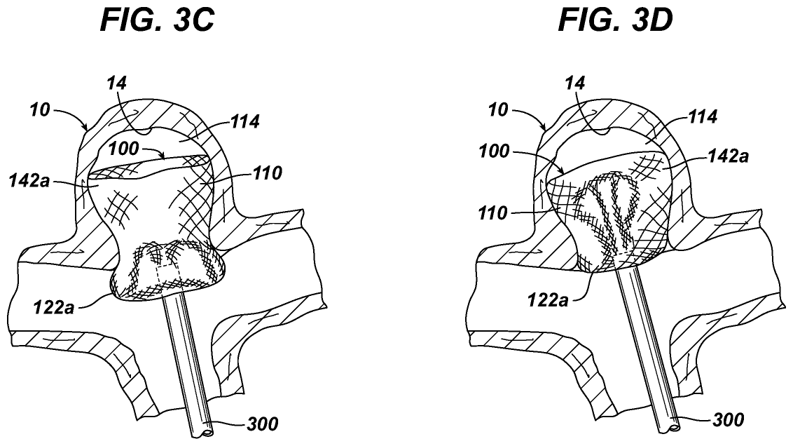

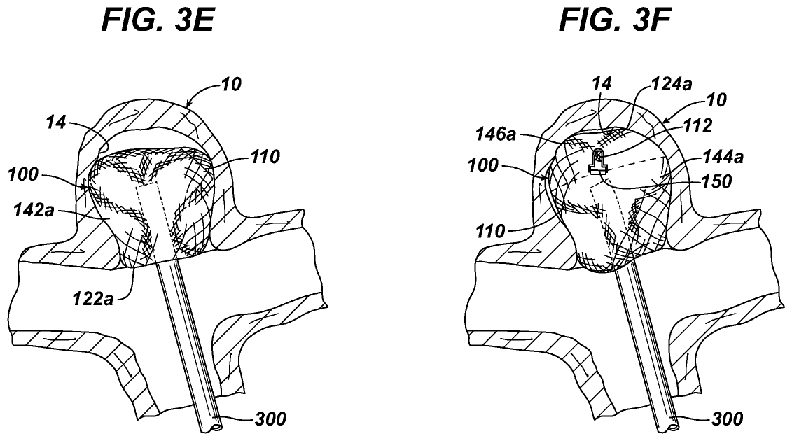

FIG. 3A through FIG. 3F is an illustration of an implant 100 having a tubular braid 110 expanding into the implanted shape. The implant 100 has a predetermined shape similar to as illustrated in FIG. 1A and can expand into the predetermined shape as illustrated in FIG. 2A through 2H but for being constrained by the shape of the aneurysm.

FIGS. 3A through 3F illustrate the implant 100 expanding from the predetermined shape into the implanted shape within an aneurysm 10. In some examples, the implant 100 can be delivered to the aneurysm 10 through the microcatheter 300. The open end 114 of the tubular braid 110 can expand within the aneurysm 10 as it exits the microcatheter 300. Over the course of its expansion, as the tubular braid 110 expands from the delivery shape into the implanted shape, the proximal inversion 122a and the distal inversion 124a are formed around the outer segments 142a, middle segment 144a, and inner segment 146a.

FIG. 3A and FIG. 3B are illustrations of the open end 114 of the tubular braid 110 positioned having exited the microcatheter 300 and expanded within the aneurysm.

FIG. 3C through FIG. 3E are a sequence of illustrations of the tubular braid 110 being pushed further distally through the microcatheter 300 and into the aneurysm 10, allowing the proximal inversion 122a to be formed and the outer segment to contact the aneurysm wall 14. In some examples, the tubular braid 110 can be configured to induce folding of the tubular braid 110 at the proximal inversion 122a during implantation of the implant 100. Due to reduced braid 110 thickness at the proximal inversion 122a, a physician is able to easily fold the tubular braid into the implanted shape without damaging the device or the aneurysm wall 14. This also allows the device to easily expand from the delivery shape into the implanted shape and provides the physician with freedom to switch between the two if necessary.

The reduced thickness and/or oxide removal at the first inversion 122 of the predetermined shape can correspond to the location of the proximal inversion 122a of the braid 110 in the implanted shape, and the increased flexibility of the proximal inversion 122a compared to the middle segment 144a can cause the proximal inversion 122a to move from within the parent blood vessel as illustrated in FIG. 3C to the neck of the aneurysm as illustrated in FIG. 3D and FIG. 3E once a sufficient length of the middle segment 144a is pushed from the distal end of the microcatheter 300. As the middle segment 144a continues to be pushed distally from the microcatheter 300, the proximal inversion 122a is at a stable position at the aneurysm neck due to the propensity of the proximal inversion 122a to create a fold in the region of reduced thickness and/or oxide removal.

FIG. 3F is an illustration of the implant 100, wherein the tubular braid 110 is fully expanded into the implanted shape. In the illustrated implanted shape, the outer segment 142a contacts the aneurysm's wall, the middle segment 144a is nested within the outer segment 142a, the inner segment 146a is positioned near the distal portion of the aneurysm wall 14, the proximal inversion 122a is positioned at the neck of the aneurysm, and the distal inversion 124a positioned near a distal portion of the aneurysm wall 14.

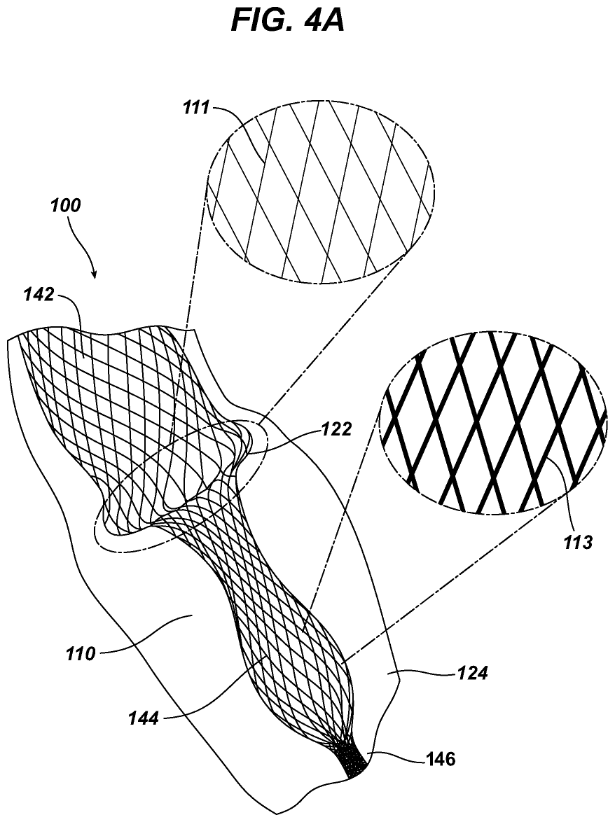

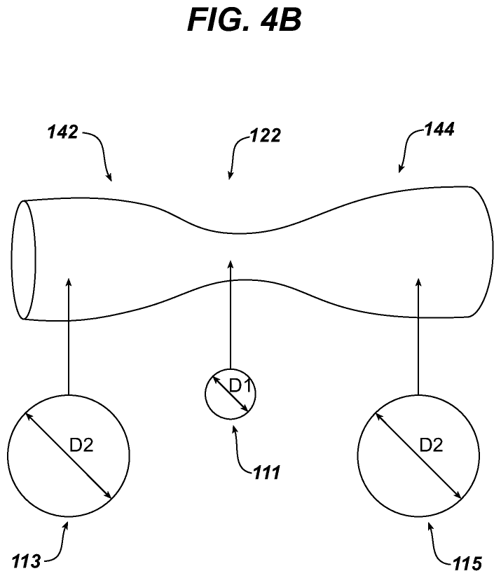

FIG. 4A is an illustration of the region of reduced wire thickness at the first inversion 122. The braid 110 has been formed into the predetermined shape (FIG. 1A) and then pulled to a single layer to form the shape illustrated in FIG. 4. The braid 110 can be compressed radially (e.g. by retracting the braid 110 into the microcatheter 300) to move the braid 110 into the delivery shape. The first segment 142 is positioned distally and has an approximately constant radius along its length, the first inversion 122 bulges outward, the second segment 144 forms a vase shape or an “S” profile, the second inversion 124 is positioned at a proximal end of the second segment 144, and the third segment 146 has a tubular shape with a small radius.

FIG. 4B is an illustration demonstrating the difference in braid thickness at the inversions of the tubular braid 110. In some examples, strands 111 of the tubular braid 110 have a smaller diameter D1 at the first inversion 122 compared to the diameter D2 of strands 113 of the first segment 142 and strands 115 of the second segment 144. The strands 111 of the tubular braid 110 can include a layer of oxide wherein the layer of oxide can be thinner at the first inversion 122 compared to an oxide layer of the first segment 142 and an oxide layer of the second segment 144.

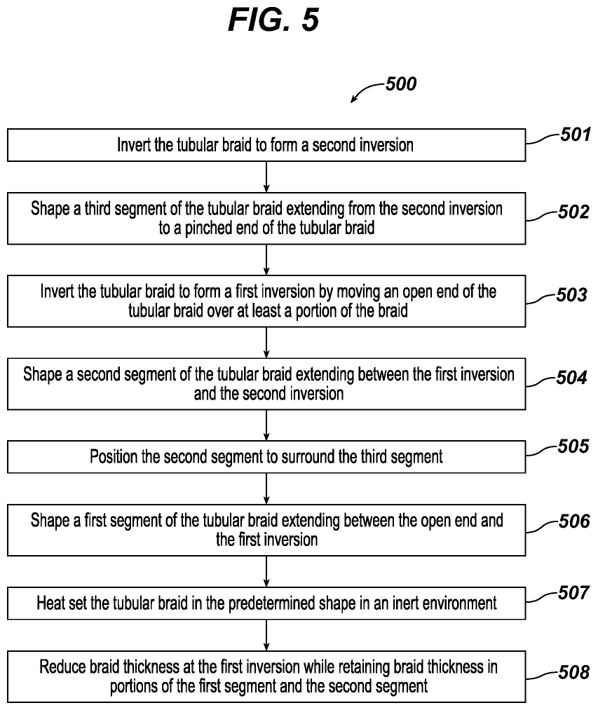

FIG. 5 shows a method 500 of manufacture for an implant 100 having a tubular braid 110 as disclosed herein. The method steps in FIG. 5 can be implemented by any of the example means described herein or by similar means, as will be appreciated. The resulting implant and tubular braid 110 can be configured similarly to the example implant 100 and tubular braid 110 presented herein, variations therefore, and alternatives thereto as understood by a person skilled in the pertinent art.

At block 501, the method 500 can include inverting the tubular braid 110 to from a second inversion 124.

At block 502, the method 500 can include shaping a third segment 146 of the tubular braid 110 extending from the second inversion 124 to a pinched end 112 of the tubular braid 110.

At block 503, the method 500 can include inverting the tubular braid 110 to form a first inversion 122 by moving an open end 114 of the tubular braid 110 over at least a portion of the braid 110.

At block 504, the method 500 can include shaping a second segment 144 of the tubular braid 110 extending between the first inversion 122 and the second inversion 124.

At block 505, the method 500 can include positioning the second segment 144 to surround the third segment 146.

At block 506, the method 500 can include shaping a first segment 142 of the tubular braid 110 extending between the open end 114 and the first inversion 122.

In some examples, blocks 501 through 506 are used to shape the tubular braid to a predetermined shape.

At block 507, the method 500 can include heat setting the tubular braid in the predetermined shape in an inert environment.

In some examples, the method 500 can further include masking at least a portion of the first segment 142 and at least a portion of the second segment 144 while exposing the first inversion 122.

At block 508, the method 500 can include reducing braid 110 thickness at the first inversion 122 while retaining braid thickness in portions of the first segment 142 and the second segment 144.

In some examples, reducing braid 110 thickness at the first inversion 122 while retaining braid 110 thickness in portions of the first segment 142 and the second segment 144 can include reducing braid 110 thickness by chemically etching or electropolishing the tubular braid 110.

In some examples, reducing braid 110 thickness at the first inversion 122 while retaining braid 110 thickness in portions of the first segment 142 and the second segment 144 can induce folding of the tubular braid 110 at the first inversion 122 during implantation of the implant 100 due to material of the tubular braid 110 being less rigid at the first inversion 122 compared to the first segment 142 and the second segment 144.

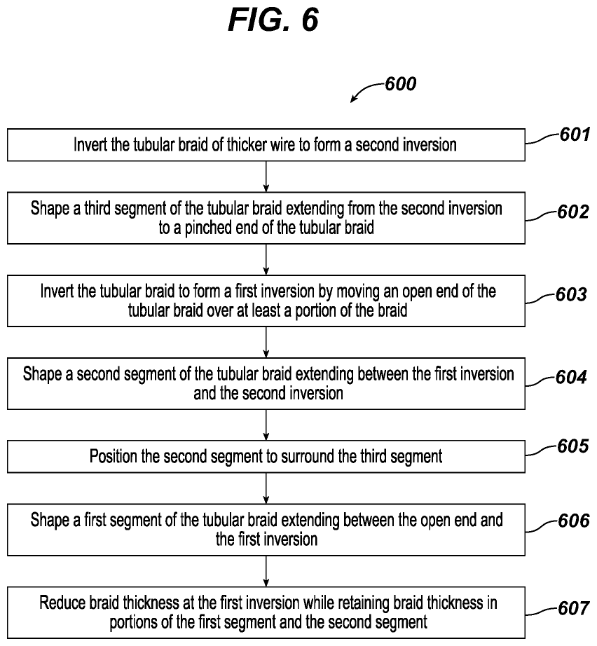

FIG. 6 shows a method 600 of manufacture for an implant 100. Method 600, different from method 500, utilizes a thicker wire. The method steps in FIG. 6 can be implemented by any of the example means described herein or by similar means, as will be appreciated. The resulting implant and tubular braid can be configured similarly to the example implant 100 and tubular braid 110 presented herein, variations therefore, and alternatives thereto as understood by a person skilled in the pertinent art.

At block 601, the method 600 can include inverting a tubular braid 110 of thicker wire to form a second inversion 124.

At block 602, the method 600 can include shaping a third segment 146 of the tubular braid 110 extending from the second inversion 124 to a pinched end 112 of the tubular braid 110.

At block 603, the method 600 can include inverting the tubular braid 110 to form a first inversion 122 by moving an open end 114 of the tubular braid 110 over at least a portion of the braid 110.

At block 604, the method 600 can include shaping a second segment 144 of the tubular braid 110 extending between the first inversion 122 and the second inversion 124.

At block 605, the method 600 can include positioning the second segment 144 to surround the third segment 146.

At block 606, the method 600 can include shaping a first segment 142 of the tubular braid 110 extending between the open end 114 and the first inversion 122.

In some examples, blocks 601 through 606 are used to shape the tubular braid of thicker wire to a predetermined shape.

At block 607, the method 600 can include reducing braid 110 thickness at the first inversion 122 while retaining braid thickness in portions of the first segment 142 and the second segment 144.

In some examples, the method 600 can further include chemically etching the tubular braid 110.

In some examples, reducing braid 110 thickness at the first inversion 122 can include masking at least a portion of the first segment 142 and at least a portion of the second segment 144 while exposing the first inversion 122.

In some examples, reducing braid 110 thickness at the first inversion 122 can further include chemically etching the tubular braid again after at least a portion of the first segment 142 and at least a portion of the second segment 144 have been masked.

In some examples, reducing braid 110 thickness at the first inversion 122 while retaining braid 110 thickness in portions of the first segment 142 and the second segment 144 can induce folding of the tubular braid 110 at the first inversion 122 during implantation of the implant 100 due to material of the tubular braid 110 being less rigid at the first inversion 122 compared to the first segment 142 and the second segment 144.

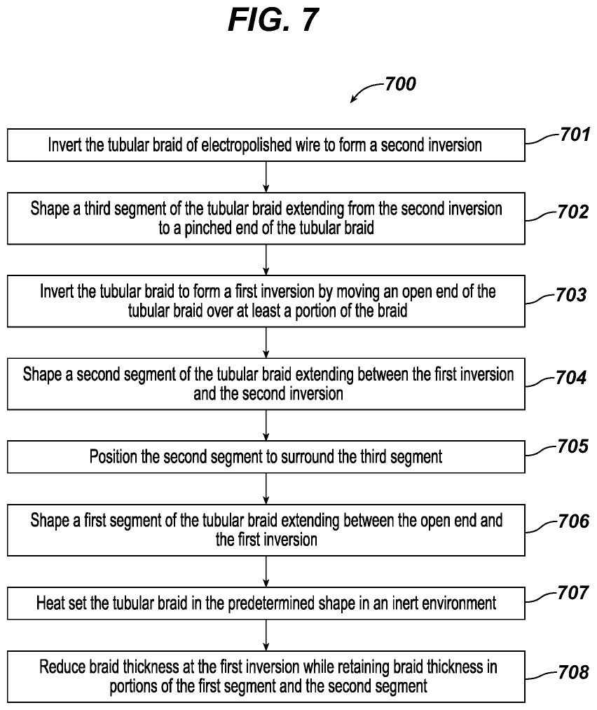

FIG. 7 shows a method 700 of manufacture for an implant 100 having a tubular braid 110 of electropolished wire as disclosed herein. The method steps in FIG. 7 can be implemented by any of the example means described herein or by similar means, as will be appreciated. The resulting implant and tubular braid can be configured similarly to the example implant 100 and tubular braid 110 presented herein, variations therefore, and alternatives thereto as understood by a person skilled in the pertinent art.

At block 701, the method 700 can include inverting a tubular braid of electropolished wire to form a second inversion 124.

At block 702, the method 700 can include shaping a third segment 146 of the tubular braid 110 extending from the second inversion 124 to a pinched end 112 of the tubular braid 110.

At block 703, the method 700 can include inverting the tubular braid 110 to form a first inversion 122 by moving an open end 114 of the tubular braid 110 over at least a portion of the braid 110.

At block 704, the method 700 can include shaping a second segment 144 of the tubular braid 110 extending between the first inversion 122 and the second inversion 124.

At block 705, the method 700 can include positioning the second segment 144 to surround the third segment 146.

At block 706, the method 700 can include shaping a first segment 142 of the tubular braid 110 extending between the open end 114 and the first inversion 122.

In some examples, blocks 701 through 706 are used to shape the tubular braid of electropolished wire to a predetermined shape.

At block 707, the method 700 can include heat setting the tubular braid in the predetermined shape in an inert environment.

In some examples, the method 700 can further include masking at least a portion of the first segment 142 and at least a portion of the second segment 144 while exposing the first inversion 122.

At block 708, the method 700 can include reducing braid 110 thickness at the first inversion 122 while retaining braid thickness in portions of the first segment 142 and the second segment 144.

In some examples, reducing braid 110 thickness at the first inversion 122 while retaining braid 110 thickness in portions of the first segment 142 and the second segment 144 can include reducing braid 110 thickness by chemically etching or electropolishing the tubular braid 110.

In some examples, reducing braid 110 thickness at the first inversion 122 while retaining braid 110 thickness in portions of the first segment 142 and the second segment 144 can induce folding of the tubular braid 110 at the first inversion 122 during implantation of the implant 100 due to material of the tubular braid 110 being less rigid at the first inversion 122 compared to the first segment 142 and the second segment 144.

Other aspects and features of the present disclosure will become apparent to those of skill in the pertinent art, upon reviewing the following detailed description in conjunction with the accompanying figures.

In describing examples, terminology is resorted to for the sake of clarity. It is intended that each term contemplates its broadest meaning as understood by those skilled in the pertinent art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device or system does not preclude the presence of additional components or intervening components between those components expressly identified.

The tubular braid 110 of the example implant 100 can include memory shape material that can be heat set to a predetermined shape, can be deformed for delivery through a catheter, and can self-expand to an implanted shape that is based on the predetermined shape and confined by the anatomy of the aneurysm in which it is implanted.

The example implant 100 described herein can rely on a radial outward force to anchor the implant within the sac of an aneurysm. To this end, the braid 110 can be shaped to a predetermined shape having a radius that is greater than its height so that the braid is radially constricted when implanted in an aneurysm. The ratio of radius to height of the braid 110 in a respective predetermined shape can be within the range of 2:1 to 1:3 to treat aneurysms of many known sizes and shapes.

The descriptions contained herein are examples of embodiments of the invention and are not intended in any way to limit the scope of the invention. As described herein, the invention contemplates many variations and modifications of the implant, including alternative materials, alternative geometries, alternative detachment features, alternative delivery systems, alternative means for forming a braid into a predetermined shape, alternative treatment methods, etc. These modifications would be apparent to those having ordinary skill in the art to which this invention relates and are intended to be within the scope of the claims which follow.

What is claimed is:

1. An implant comprising:
a tubular braid comprising an open end, a pinched end, and a predetermined shape,
wherein, in the predetermined shape, the tubular braid comprises a first segment extending from the open end to a first inversion, a second segment extending from the first inversion to a second inversion, and a third segment surrounded by the second segment and extending from the second inversion to the pinched end, and
wherein, strands of the tubular braid have a smaller diameter at the first inversion compared to a strand diameter of the first segment and a strand diameter of the second segment.

2. The implant of claim 1, wherein material of the tubular braid at the first inversion is weakened by oxide removal.

3. The implant of claim 1,
wherein strands of the tubular braid comprise a layer of oxide, and
wherein the layer of oxide is thinner at the first inversion compared to an oxide layer of the first segment and an oxide layer of the second segment.

4. The implant of claim 1, wherein the smaller diameter of the strands at the first inversion is configured to induce folding of the tubular braid at the first inversion during implantation of the implant.

5. The implant of claim 1,
wherein the tubular braid can be configured to expand from the predetermined shape into an implanted shape, and
wherein the smaller diameter at the first inversion configured to induce folding of the tubular braid allows this expansion.

6. A method comprising:
shaping a tubular braid to a predetermined shape comprising:
inverting the tubular braid to form a second inversion,
shaping a third segment of the tubular braid extending from the second inversion to a pinched end of the tubular braid,
inverting the tubular braid to form a first inversion by moving an open end of the tubular braid over at least a portion of the braid,
shaping a second segment of the tubular braid extending between the first inversion and the second inversion,
positioning the second segment to surround the third segment, and
shaping a first segment of the tubular braid extending between the open end and the first inversion;
heat setting the tubular braid in the predetermined shape in an inert environment; and
reducing braid thickness at the first inversion while retaining braid thickness in portions of the first segment and the second segment.

7. The method of claim 6, wherein reducing braid thickness at the first inversion while retaining braid thickness in portions of the first segment and the second segment comprises masking at least a portion of the first segment and at least a portion of the second segment while exposing the first inversion.

8. The method of claim 6, wherein reducing braid thickness at the first inversion while retaining braid thickness in portions of the first segment and the second segment comprises chemically etching the tubular braid.

9. The method of claim 6, wherein reducing braid thickness at the first inversion while retaining braid thickness in portions of the first segment and the second segment comprises electropolishing the tubular braid.

10. The method of claim 6, wherein reducing braid thickness at the first inversion while retaining thickness in portions of the first segment and the second segment results in material of the tubular braid being less rigid at the first inversion compared to the first segment and the second segment in order to induce folding of the tubular braid at the first inversion during implantation of the implant.

11. A method comprising:
shaping a tubular braid of thicker wire to a predetermined shape comprises:
inverting the tubular braid to form a second inversion;
shaping a third segment of the tubular braid extending from the second inversion to a pinched end of the tubular braid;
inverting the tubular braid to form a first inversion by moving an open end of the tubular braid over at least a portion of the braid;
shaping a second segment of the tubular braid extending between the first inversion and the second inversion;
positioning the second segment to surround the third segment, and
shaping a first segment of the tubular braid extending between the open end and the first inversion; and
reducing braid thickness at the first inversion while retaining braid thickness in portions of the first segment and the second segment.

12. The method of claim 11, wherein reducing braid thickness at the first inversion while retaining braid thickness in portions of the first segment and the second segment comprises chemically etching the tubular braid.

13. The method of claim 11, wherein reducing braid thickness at the first inversion while retaining braid thickness in portions of the first segment and the second segment further comprises masking at least a portion of the first segment and at least a portion of the second segment while exposing the first inversion.

14. The method of claim 11, wherein reducing braid thickness at the first inversion while retaining braid thickness in portions of the first segment and the second segment further comprises chemically etching the tubular braid again after at least a portion of the first segment and at least a portion of the second segment have been masked.

15. The method of claim 11, wherein reducing braid thickness at the first inversion while retaining thickness in portions of the first segment and the second segment results in material of the tubular braid being less rigid at the first inversion compared to the first segment and the second segment in order to induce folding of the tubular braid at the first inversion during implantation of the implant.

16. A method comprising:
shaping a tubular braid of electropolished wire to a predetermined shape comprising the steps of:
inverting the tubular braid to form a second inversion;
shaping a third segment of the tubular braid extending from the second inversion to a pinched end of the tubular braid;
inverting the tubular braid to form a first inversion by moving an open end of the tubular braid over at least a portion of the braid;
shaping a second segment of the tubular braid extending between the first inversion and the second inversion;
positioning the second segment to surround the third segment, and
shaping a first segment of the tubular braid extending between the open end and the first inversion;
heat setting the tubular braid in the predetermined shape in an inert environment; and
reducing braid thickness at the first inversion while retaining braid thickness in portions of the first segment and the second segment.

17. The method of claim 16, wherein reducing braid thickness at the first inversion while retaining braid thickness in portions of the first segment and the second segment comprises masking at least a portion of the first segment and at least a portion of the second segment while leaving the first inversion exposed.

18. The method of claim 16, wherein reducing braid thickness at the first inversion while retaining braid thickness in portions of the first segment and the second segment comprises chemically etching the tubular braid.

19. The method of claim 16, wherein reducing braid thickness at the first inversion while retaining braid thickness in portions of the first segment and the second segment comprises electropolishing the tubular braid.

20. The method of claim 16, wherein reducing braid thickness at the first inversion while retaining thickness in portions of the first segment and the second segment results in material of the tubular braid being less rigid at the first inversion compared to the first segment and the second segment in order to induce folding of the tubular braid at the first inversion during implantation of the implant.
